(19) United States
(12) Patent Application Publication
(10) Pub. No.: US 2024/0376032 A1
(43) Pub. Date: Nov. 14, 2024

(54) METHOD FOR REDUCING 1,1,1,2,2-PENTAFLUOROPROPANE (HFC-245CB) IN A PROCESS FOR PRODUCING TRANS-1,3,3,3-TETRAFLUOROPROPENE (HFO-1234ZE(E))

(71) Applicant: Honeywell International Inc., Charlotte, NC (US)

(72) Inventors: Richard D. Horwath, Richmond, VA (US); Gustavo Cerri, Parsippany, NJ (US); Benson May, Charlotte, NC (US); Haiyou Wang, Amherst, NY (US)

(21) Appl. No.: 18/660,212

(22) Filed: May 9, 2024

Related U.S. Application Data
(60) Provisional application No. 63/466,119, filed on May 12, 2023.

(51) Int. Cl. C07C 17/25; B01J 23/26; C07C 17/383

(52) U.S. Cl. CPC C07C 17/25; B01J 23/26; C07C 17/383

(57) ABSTRACT

A method for reducing 1,1,1,2,2-pentafluoropropane (HFC-245cb) in a trans-1,3,3,3-tetrafluoropropene (HFO-1234ze(E)) manufacturing process including catalyst conditioning, temperature reduction, and contact time reduction. The method includes dehydrofluorinating 1,1,1,3,3-pentafluoropropane (HFC-245fa) with a catalyst mixture to produce a product mixture with HFO-1234ze(E) and HFC-245cb, and either incorporating a conditioned catalyst, using a lower temperature for the dehydrofluorination, or reducing the contact time for the dehydrofluorination to reduce the formation of HFC-245cb.

### CROSS-REFERENCE TO RELATED APPLICATIONS

This application claims the benefit under 35 U.S.C. 119(e) of U.S. Provisional Patent Application No. 63/466,119, filed May 12, 2023, which is herein incorporated by reference in its entirety.

### FIELD

The present disclosure relates to a process for manufacturing trans-1,3,3,3-tetrafluoropropene (HFO-1234ze(E)), and specifically to a method for reducing the production of 1,1,1,2,2-pentafluoropropane (HFC-245cb) in the HFO-1234ze(E) manufacturing process.

### BACKGROUND

Chlorofluorocarbons (CFCs) like trichlorofuoromethane and dichlorodifluoromethane have been used as refrigerants, blowing agents and diluents for gaseous sterilization. In recent years, there has been widespread concern that certain chlorofluorocarbons might be detrimental to the Earth's ozone layer. As a result, there is a worldwide effort to use halocarbons which contain fewer or no chlorine substituents. Accordingly, the production of hydrofluorocarbons, or compounds containing only carbon, hydrogen and fluorine, has been the subject of increasing interest to provide environmentally desirable products for use as solvents, blowing agents, refrigerants, cleaning agents, aerosol propellants, heat transfer media, dielectrics, fire extinguishing compositions and power cycle working fluids. In this regard, trans-1,3,3,3-tetrafluoropropene (HFO-1234ze(E)) is a compound that has the potential to be used as a zero Ozone Depletion Potential (ODP) and a low Global Warming Potential (GWP) refrigerant, blowing agent, aerosol propellant, solvent, etc, and also as a fluorinated monomer.

It has been determined that methods for the production of HFO-1234ze(E) are sometimes not economical relative to their product yield because of impurities present in the HFO-1234ze(E) product stream. Certain applications, such as medical propellants, require extremely high purity HFO-1234ze(E). It has been noted that, among other impurities, significant amounts of 1,1,1,2,2-pentafluoropropane (HFC-245cb) may be generated together with the desired product. Accordingly, the present disclosure provides an integrated process for reducing the production of HFC-245cb in the HFO-1234ze(E) manufacturing process.

### SUMMARY

The present disclosure is based on the discovery that certain modifications to the HFO-1234ze(E) process may significantly reduce the production of HFC-245cb. Advantageously, these modifications may be done without significant structural disruption to existing HFO-1234ze(E) process reactors and eliminate the need for subjecting the crude HFO-1234ze(E) product stream to costly and time-consuming separations.

In one form thereof, the present disclosure provides a method for reducing 1,1,1,2,2-pentafluoropropane (HFC-245cb) in a trans-1,3,3,3-tetrafluoropropene (HFO-1234ze(E)) manufacturing process comprising: dehydrofluorinating 1,1,1,3,3-pentafluoropropane (HFC-245fa) with a catalyst mixture to produce a product mixture comprising trans-1,3,3,3-tetrafluoropropene (HFO-1234ze(E)), cis-1,3,3,3-tetrafluoropropene (HFO-1234ze(Z)), 1,1,1,2,2-pentafluoropropane (HFC-245cb), and hydrogen fluoride (HF), wherein the catalyst mixture comprises from 10 wt. % to 90 wt. % of a conditioned catalyst having a runtime of from 20 to 500 days in the HFO-1234ze(E) manufacturing process, based on the total weight of the catalyst mixture.

In another form thereof, the present disclosure provides a method for reducing of 1,1,1,2,2-pentafluoropropane (HFC-245cb) in a trans-1,3,3,3-tetrafluoropropene (HFO-1234ze(E)) manufacturing process comprising: dehydrofluorinating 1,1,1,3,3-pentafluoropropane (HFC-245fa) with a catalyst mixture to produce a product mixture comprising trans-1,3,3,3-tetrafluoropropene (HFO-1234ze(E)), cis-1,3,3,3-tetrafluoropropene (HFO-1234ze(Z)), 1,1,1,2,2-pentafluoropropane (HFC-245cb), and hydrogen fluoride (HF), wherein the dehydrofluorination step is conducted at a temperature of from 10° C. to 310° C.

In another form thereof, the present disclosure provides a method for reducing 1,1,1,2,2-pentafluoropropane (HFC-245cb) in a trans-1,3,3,3 tetrafluoropropene (HFO-1234ze(E)) manufacturing process comprising: dehydrofluorinating 1,1,1,3,3-pentafluoropropane (HFC-245fa) with a catalyst mixture to produce a product mixture comprising trans-1,3,3,3-tetrafluoropropene (HFO-1234ze(E)), cis-1,3,3,3-tetrafluoropropene (HFO-1234ze(Z)), 1,1,1,2,2-pentafluoropropane (HFC-245cb), and hydrogen fluoride (HF), wherein the contact time in the dehydrofluorination step is from 1 second to 40 seconds.

### BRIEF DESCRIPTION OF THE DRAWINGS

The above mentioned and other features of the disclosure, and the manner of attaining them, will become more apparent and the disclosure itself will be better understood by reference to the following description of embodiments of the disclosure taken in conjunction with the accompanying drawings.

FIG. 1 is a process flow diagram for the HFO-1234ze(E) manufacturing process.

The exemplification set out herein illustrates an embodiment of the disclosure, and such exemplification is not to be construed as limiting the scope of the disclosure in any manner.

### DETAILED DESCRIPTION

### I. Definitions

By way of non-limiting illustration, examples of certain embodiments of the present disclosure are given below.

As used herein, the term “HFO-1234ze(E)” refers to the trans isomer of 1,3,3,3-tetrafluoropropene.

The term “HFO-1234ze(Z)” refers to the cis isomer of 1,3,3,3-tetrafluoropropene.

The term “HFC-245cb” refers to 1,1,1,2,2-pentafluoropropane.

The term “HFC-245fa” refers to 1,1,1,3,3-pentafluoropropane.

The term “fresh catalyst” as used herein refers to a catalyst with 0 days of runtime in the HFO-1234ze(E) manufacturing process, i.e., an “unused” catalyst.

The term “conditioned catalyst” as used herein refers to a catalyst with at least one day (24 hours) of runtime.

### II. Integrated Process for the Production of HFO-1234Ze(E)

A process for the production of HFO-1234ze(E) is described in detail in U.S. Pat. No. 7,638,660B2 which is incorporated by reference in its entirety.

The process may include the following steps:
(a) dehydrofluorinating 1,1,1,3,3-pentafluoropropane (HFC-245fa) with a catalyst mixture to produce a product mixture comprising trans-1,3,3,3-tetrafluoropropene (HFO-1234ze(E)), cis-1,3,3,3-tetrafluoropropene (HFO-1234ze(Z)), 1,1,1,2,2-pentafluoropropane (HFC-245cb), and hydrogen fluoride (HF)
(b) optionally recovering hydrogen fluoride from the result of step (a);
(c) optionally isomerizing at least a portion of the cis-1,3,3,3-tetrafluoropropene into trans-1,3,3,3-tetrafluoropropene;
(d) recovering the reaction product mixture and recycling it back into the dehydrofluorination step; and
(e) recovering trans-1,3,3,3-tetrafluoropropene from the product mixture, optionally by distillation.

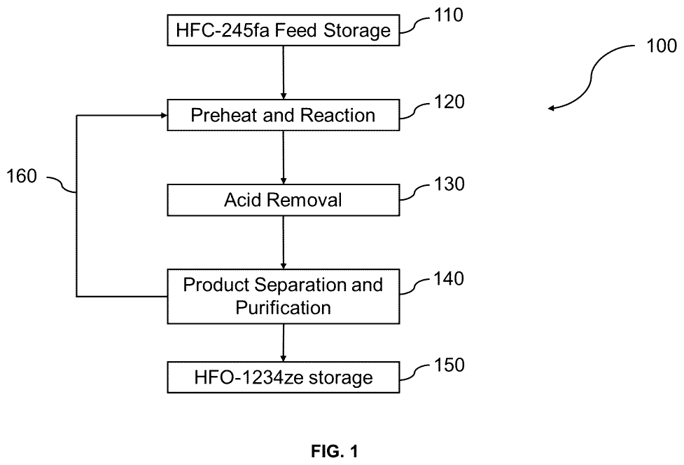

Referring to FIG. 1, showing an exemplary flow diagram for the production of HFO-1234ze(E), in a first step HFC-245fa feed 110 is provided. The HFC-245fa feed is then preheated and reacted in a reaction step 120. In the reaction step 120, HFC-245fa is dehydrofluorinated by catalytic conversion of HFC-245fa yielding a product stream including cis-1,3,3,3-tetrafluoropropene, trans-1,3,3,3-tetrafluoropropene and hydrogen fluoride. Optionally hydrogen fluoride may be recovered from product stream in an acid removal step 130. Following acid removal, the reaction product may be purified in a product purification step 140. The purified HFO-1234ze may be stored in step 150. Optionally, the mixture of HFO-1234ze(Z) and HFC-245fa recovered as a distillate, may be recycled to the reaction step 120 via recycle stream 160.

Generally, dehydrofluorination reactions are well known in the art. Preferably, the dehydrofluorination of HFC-245fa is conducted in a vapor phase, and more preferably in a fixed-bed reactor in the vapor phase. The dehydrofluorination reaction may be conducted in any suitable reaction vessel or reactor, but it should preferably be constructed from materials which are resistant to the corrosive effects of hydrogen fluoride such as nickel and its alloys, including Hastelloy, Inconel, Incoloy, and Monel or vessels lined with fluoropolymers. These may be single pipe or multiple tubes packed with a dehydrofluorinating catalyst which may be one or more of fluorinated metal oxides in bulk form or supported, metal halides in bulk form or supported, and carbon supported transition metals, metal oxides and halides.

Suitable catalysts non-exclusively include fluorinated chromia (fluorinated Cr₂O₃), fluorinated alumina (fluorinated Al₂O₃), fluorinated mixed metal oxides (e.g., ZnO—Cr₂O₃), metal fluorides (e.g., CrF₃, AlF₃) and carbon supported transition metals (zero oxidation state) such as Fe/C, Co/C, Ni/C, Pd/C.

For example, suitable catalysts may include chromium oxides, chromium oxyfluorides, and chromium halides. The chromium oxides may include amorphous chromium oxide (Cr₂O₃), crystalline chromium oxide, and combinations of the foregoing. The chromium oxyfluorides may include fresh amorphous chromium oxide (Cr₂O₃) pretreated with HF, fresh crystalline chromium oxide (Cr₂O₃) pretreated with HF, amorphous chromium oxyfluoride (CrOₓF_{y}, where x may be greater than 0 but less than 1.5, and y may be greater than 0 but less than 3), crystalline chromium oxyfluoride (CrOₓF_{y}, where x may be greater than 0 but less than 1.5, and y may be greater than 0 but less than 3), and combinations of the foregoing. In one embodiment, the catalyst is amorphous chromium oxyfluoride (CrOₓF_{y}, where x may be greater than 0 but less than 1.5, and y may be greater than 0 but less than 3). The chromium halides may include chromium trifluoride (CrF₃), chromium trichloride (CrCl₃), chromium triiodide (Crl₃) and chromium tribromide (CrBr₃), and combinations of the foregoing. In one embodiment, the catalyst is chromium trifluoride (CrF₃).

Other suitable catalysts include promoted chromium-based catalysts, which are based on chromium and include an amount of at least one co-catalyst selected from Ni, Zn, Co, Mn, Mg, or mixtures thereof. The amount of the co-catalyst may be between 0.1 wt. % and 20 wt. % based on the total weight of the catalyst and, more particularly, may be present in an amount as little as 0.1 wt. %, 0.5 wt. %, 1.0 wt. % 1.5 wt. % or as high as 2.0 wt. %, 3.0 wt. %, 4.0 wt. %, 5.0 wt. %, 6.0 wt. %, or within any range using any two of the foregoing values as endpoints, based on to total weight of the catalyst. One suitable promoted chromium catalyst is a zinc/chromia catalyst which is based on chromia and includes an amount of zinc as a co-catalyst, for example, JM 62-3M catalyst available from Johnson Matthey. Prior to use, a fluorination treatment of the catalyst may be conducted using anhydrous HF under conditions effective to convert a portion of metal oxides into corresponding metal fluorides.

The above chromium-based catalysts may also be low chromium (VI) catalysts, having a total content of chromium (VI) oxide in an amount of about 5,000 ppm or less, about 2,000 ppm or less, about 1,000 ppm or less, about 500 ppm or less, about 250 ppm or less, or about 100 ppm or less based on total chromium oxides in the chromium oxide catalyst.

In addition to chromium-based catalysts, other suitable catalysts include alumina, iron oxide, magnesium oxide, zinc oxide, nickel oxide, cobalt oxide, aluminum fluoride or metal fluorides such as iron fluoride, magnesium fluoride, zinc fluoride, nickel fluoride, cobalt fluoride, fluorinated alumina, fluorinated iron oxide, fluorinated magnesium oxide, fluorinated nickel oxide, fluorinated cobalt oxide, titanium fluorides, molybdenum fluorides, aluminum oxyfluorides, and combinations of the foregoing. Prior to use, a fluorination treatment of catalyst containing metal oxide(s) is conducted using anhydrous HF under conditions effective to convert a portion of metal oxide(s) into corresponding metal fluoride(s).

The HFC-245fa is introduced into the reactor either in pure form, impure form, or together with an optional inert gas diluent such as nitrogen, argon, or the like. In a preferred embodiment of the invention, the HFC-245fa is pre-vaporized or preheated prior to entering the reactor. Alternatively, the HFC-245fa is vaporized inside the reactor. Useful reaction temperatures may range from about 100° C. to about 600° C. Preferred temperatures may range from about 150° C. to about 450° C., and more preferred temperatures may range from about 200° C. to about 350° C. The reaction may be conducted at atmospheric pressure, super-atmospheric pressure or under vacuum. The vacuum pressure can be from about 5 torr to about 760 torr. Contact time of the HFC-245fa with the catalyst may range from about 0.5 seconds to about 120 seconds, however, longer or shorter times can be used.

In the preferred embodiment, the process flow is in the down or up direction through a bed of the catalyst. It may also be advantageous to periodically regenerate the catalyst after prolonged use while in place in the reactor. Regeneration of the catalyst may be accomplished by any means known in the art, for example, by passing air or air diluted with nitrogen over the catalyst at temperatures of from about 100° C. to about 400° C., preferably from about 200° C. to about 375° C., for from about 0.5 hour to about 3 days. This is followed by either HF treatment at temperatures of from about 25° C. to about 400° C., preferably from about 200° C. to about 350° C. for fluorinated metal oxide catalysts and metal fluoride ones or H₂ treatment at temperatures of from about 100° C. to about 400° C., preferably from about 200° C. to about 350° C. for carbon supported transition metal catalysts.

In an alternative embodiment of the invention, dehydrofluorination of HFC-245fa can also be accomplished by reacting it with a strong caustic solution that includes, but is not limited to KOH, NaOH, Ca(OH)₂ and CaO at an elevated temperature. In this case, the caustic strength of the caustic solution is of from about 2 wt. % to about 100 wt. %, more preferably from about 5 wt. % to about 90 wt. % and most preferably from about 10 wt. % to about 80 wt. %. The reaction may be conducted at a temperature of from about 20° C. to about 100° C., more preferably from about 30° C. to about 90° C. and most preferably from about 40° C. to about 80° C. As above, the reaction may be conducted at atmospheric pressure, super-atmospheric pressure or under vacuum. The vacuum pressure can be from about 5 torr to about 760 torr. In addition, a solvent may optionally be used to help dissolve the organic compounds in the caustic solution. This optional step may be conducted using solvents that are well known in the art for said purpose.

Recovering of hydrogen fluoride is conducted by passing the composition resulting from the dehydrofluorination reaction through a sulfuric acid extractor to remove hydrogen fluoride, subsequently desorbing the extracted hydrogen fluoride from the sulfuric acid, and then distilling the desorbed hydrogen fluoride. The separation may be conducted by adding sulfuric acid to the mixture while the mixture is in either the liquid or gaseous states. The usual weight ratio of sulfuric acid to hydrogen fluoride ranges from about 0.1:1 to about 100:1. One may begin with a liquid mixture of the fluorocarbons and hydrogen fluoride and then add sulfuric acid to the mixture.

The amount of sulfuric acid needed for the separation depends on the amount of HF present in the system. From the solubility of HF in 100% sulfuric acid as a function of a temperature curve, the minimum practical amount of sulfuric acid can be determined. For example at 30° C., about 34 g of HF will dissolve in 100 g of 100% sulfuric acid. However, at 100° C., only about 10 g of HF will dissolve in the 100% sulfuric acid. Preferably the sulfuric acid used in this invention has a purity of from about 50% to 100%.

In the preferred embodiment, the weight ratio of sulfuric acid to hydrogen fluoride ranges from about 0.1:1 to about 1000:1. More preferably the weight ratio ranges from about 1:1 to about 100:1 and most preferably from about 2:1 to about 50:1. Preferably the reaction is conducted at a temperature of from about 0° C. to about 100° C., more preferably from about 0° C. to about 40° C., and most preferably from about 20° C. to about 40° C. The extraction is usually conducted at normal atmospheric pressure; however, higher or lower pressure conditions may be used by those skilled in the art. Upon adding the sulfuric acid to the mixture of fluorocarbons and HF, two phases rapidly form.

An upper phase is formed which is rich in the fluorocarbons and a lower phase which is rich in HF/sulfuric to acid. The term “rich” means the phase contains more than 50% of the indicated component in that phase, and preferably more than 80% of the indicated component in that phase. The extraction efficiency of the fluorocarbon can range from about 90% to about 99%.

After the separation of the phases, one removes the upper phase rich in the fluorocarbons from the lower phase rich in the hydrogen fluoride and sulfuric acid. This may be done by decanting, siphoning, distillation or other techniques well known in the art. One may optionally repeat the fluorocarbon extraction by adding more sulfuric acid to the removed lower phase. With about a 2.25:1 weight ratio of sulfuric acid to hydrogen fluoride, one can obtain an extraction efficiency of about 92% in one step. Preferably one thereafter separates the hydrogen fluoride and sulfuric acid. One can take advantage of the low solubility of HF in sulfuric at high temperatures to recover the HF from sulfuric. For example, at 140° C., only 4 g of HF will dissolve in 100% sulfuric acid. One can heat the HF/sulfuric acid solution up to 250° C. to recover the HF. The HF and sulfuric acid may then be recycled. That is, the HF may be recycled to a preceding reaction for the formation of the HFC-245fa and the sulfuric acid may be recycled for use in further extraction steps.

In another embodiment of the invention, the recovering of hydrogen fluoride from the mixture of fluorocarbon and hydrogen fluoride may be conducted in a gaseous phase by a continuous process of introducing a stream of sulfuric acid to a stream of fluorocarbon and hydrogen fluoride. This may be conducted in a standard scrubbing tower by flowing a stream of sulfuric acid countercurrent to a stream of fluorocarbon and hydrogen fluoride. Sulfuric acid extraction is described, for example in U.S. Pat. No. 5,895,639, which is incorporated herein by reference.

Alternatively, HF can be recovered or removed by using water or caustic scrubbers, or by contacting with a metal salt. When water extractor is used, the technique is similar to that of sulfuric acid. When caustic is used, HF is removed from system as a fluoride salt in aqueous solution. When metal salt (e.g. potassium fluoride, or sodium fluoride) is used, it can be used neat or in conjunction with water. HF can be recovered when metal salt is used. HF can also be recovered by adsorption in water followed by azeotropic distillation of the HF/water solution to recover anhydrous HF.

Trans-1,3,3,3-tetrafluoropropene may be recovered from the reaction product mixture comprised of unreacted starting materials and by-products, including cis-1,3,3,3-tetrafluoropropene and any by-products and/or starting materials by any means known in the art, such as by extraction and preferably distillation. The mixture of trans-1,3,3,3-tetrafluoropropene, cis-1,3,3,3-tetrafluoropropene, unreacted HFC-245fa and any by-products are passed through a distillation column. For example, the distillation may be preferably conducted in a standard distillation column at atmospheric pressure, super-atmospheric pressure or a vacuum. Preferably the pressure is less than about 300 psig, more preferably less than about 150 psig and most preferably less than 100 psig. The pressure of the distillation column inherently determines the distillation operating temperature. Trans-1,3,3,3-tetrafluoropropene has a boiling point of about −19° C.; cis-1,3,3,3-tetrafluoropropene has a boiling point of about 9° C.; HFC-245fa has a boiling point of about 15° C. Trans-1,3,3,3-tetrafluoropropene may be recovered as distillate by operating the distillation column at from about −10° C. to about 90° C., preferably from about 0° C. to about 80° C. Single or multiple distillation columns may be used. The distillate portion includes substantially all the trans-1,3,3,3-tetrafluoropropene. The bottom stream of the distillation includes cis-1,3,3,3-tetrafluoropropene, HFC-245fa, as well as any other impurities such as HFC-1233zdE/Z, CFC-113, and various dimers and trimers. The bottom stream can be optionally further distilled by using another distillation column to recover a recyclable stream comprising HFC-245fa, HFO-1234zeZ, HCFO-1233zdE/Z for recycle and to remove undesired impurities including CFC-113 and various dimers and trimers.

Then at least a portion of the cis-1,3,3,3-tetrafluoropropene (HFO-1234ze(Z)) is isomerized into trans-1,3,3,3-tetrafluoropropene (HFO-1234ze(Z)). A stream of cis-1,3,3,3-tetrafluoropropene or its mixture with trans-1,3,3,3-tetrafluoropropene and/or 1,1,1,3,3-pentafluoropropane is fed into an isomerization reactor which contains a suitable isomerization catalyst (e.g., fluorinated metal oxides in bulk or supported, metal fluorides in bulk or supported, carbon supported transition metals, etc.) to convert most of the HFO-1234ze(Z) into HFO-1234ze(E). The isomerization reaction may be conducted in any suitable reaction vessel or reactor, but it should preferably be constructed from materials which are resistant to corrosion such as nickel and its alloys, including Hastelloy, Inconel, Incoloy, and Monel or vessels lined with fluoropolymers. These may be single pipe or multiple tubes packed with an isomerization catalyst which may be a fluorinated metal oxide, metal fluoride, or a carbon supported transition metal. Suitable catalysts non-exclusively include fluorinated chromia, chromium fluoride, fluorinated ZnO—Cr₂O₃, fluorinated alumina, aluminum fluoride, and carbon supported cobalt. Useful reaction temperatures may range from about 25° C. to about 450° C. Preferred temperatures may range from about 50° C. to about 350° C., and more preferred temperatures may range from about 75° C. to about 250° C. The reaction may be conducted at atmospheric pressure, super-atmospheric pressure or under vacuum. The vacuum pressure can be from about 5 torr to about 760 torr. Contact time of the cis-1,3,3,3-tetrafluoropropene with the catalyst may range from about 0.5 seconds to about 120 seconds, however, longer or shorter times can be used. In a preferred scenario, the HFO-1234ze(Z) isomerization reaction and the HFC-245fa dehydrofluorination reaction take place in the same reactor charged with a fluorinated chromia catalyst or a fluorinated ZnO—Cr₂O₃ catalyst.

In the following alternative embodiments of the invention, the HFC-245fa dehydrofluorination reactor and the HFO-1234ze(Z) isomerization reactor can be combined or independent. The HFO-1234ze(E) isolation can be after or before the HFO-1234ze(Z) isomerization reaction.

### Alternative 1:

(1) Combined reaction of HFC-245fa dehydrofluorination and HFO-1234ze(Z) isomerization in one reaction vessel.
(2) Optional HF recovery.
(3) Isolation of HFO-1234ze(E). Optionally, the remaining mixture is recycled back to step 1.
(4) Optional distillation of recycle mixture to remove high-boiling components such as HCFC-113 and HCFO-1233zd.

### Alternative 2:

(1) Catalytic dehydrofluorination of HFC-245fa into a composition comprising HFO-1234ze(Z).
(2) Optional HF recovery.
(3) Isolation of HFO-1234ze(E) wherein the outlet stream of (2) feeds into a distillation column. The product, HFO-1234ze(E), is isolated as a distillate from the rest of the mixture, i.e. HFO-1234ze(Z), the un-reacted HFC-245fa and other minor by-products. The residual amounts of HF/HCl present in the distillate are removed and followed by an acid-removal step. The bottom stream from the distillation of (3) is split to two streams and fed to steps (4) and (1), respectively. Optionally, further distillation is conducted by using another distillation column after step (3). In this distillation column, the mixture of HFO-1234ze(Z) and HFC-245fa is recovered as a distillate, which is subsequently fed to step (4). The bottom stream from this 2nd distillation column is optionally recycled back to step (1).

### (4) Catalytic Isomerization of HFO-1234Ze(Z).

The mixture of HFO-1234ze(Z)/HFC-245fa from step (3) is fed into an isomerization reactor which contains a suitable isomerization catalyst to convert most of the HFO-1234ze(Z) into HFO-1234ze(E). The effluent from the catalytic reactor of step (4) is fed into step (3) for HFO-1234ze(E) isolation.

### Alternative 3:

(1) Catalytic dehydrofluorination of 245fa into trans/HFO-1234ze(Z).
(2) Optional HF recovery.
(3) Isolation of HFO-1234ze(E)
(4) Catalytic isomerization of HFO-1234ze(Z) wherein the mixture comprising HFO-1234ze(Z) and 245fa from step (3) is fed into an isomerization reactor which contains a suitable isomerization catalyst to convert most of the HFO-1234ze(Z) into HFO-1234ze(E).
(5) Isolation of HFO-1234ze(E) wherein the effluent from step (4) is fed into a distillation column. The product, HFO-1234ze(E), is isolated as a distillate from the rest of the mixture, i.e. HFO-1234ze(Z), the un-reacted 245fa and other minor by-products. The bottom stream from the distillation of (5) is recycled back to step (1).

During the manufacture of HFO-1234ze(E), undesirable impurities such as HFC-245cb may be produced. Section III below discusses methods for reducing the production of HFC-245cb.

### III. Methods for Reducing Production of HFC-245cb

HFC-245cb is a hydrofluorocarbon which is produced as an impurity in the HFO-1234ze(E) manufacturing process. It is an isomer of the starting material HFC-245fa, shown below.

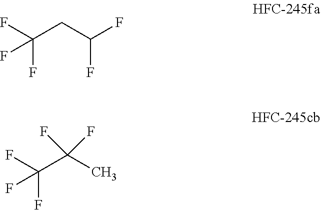

The production of HFC-245cb is problematic because it forms a light-boiling azeotrope with the desired product, HFO-1234ze(E).

There is currently a need for very high purity HFO-1234ze(E) for medical propellants such as for inhalers. The desire for production of high purity HFO-1234ze(E) may be met by several methods. A crude product stream comprising HFO-1234ze(E) may be purified via distillation or other separation methods to remove HFC-245cb. These separations, while effective, introduce complexity, cost, and added time to the manufacturing process. Alternatively, the manufacturing process may be modified to prevent or mitigate the reactions which form HFC-245cb in the first place.

The present disclosure contemplates three methods for reducing the production of HFC-245cb in the HFO-1234ze(E) process: catalyst conditioning, reducing temperature, and reducing contact time. These methods are designed to preempt the production of high levels of HFC-245cb in the reactor so that later separations are either more efficient or not needed at all.

### A. Catalyst Conditioning

In the HFO-1234ze(E) process outlined in Section II, the catalyst may be changed out periodically either due to loss of activity (i.e. low conversion of 245fa or high reactor temperature required for adequate 245fa conversion) or the need to conduct an internal inspection of the reactor.

It has been found that the renewal of the catalyst produces a spike in the production of the HFC-245cb impurity. The production of HFC-245cb may be produced at levels as high as 1000 ppm when a full batch of fresh catalyst is introduced into the reactor. Without being bound to theory, it is believed the conversion of 245fa to 245cb takes place via the following multiple steps: CF₃CH₂CHF₂ (245fa)→CF₃CH=CHF+HF, CF₃CH=CHF+CF₃CCH+HF, CF₃CCH+HF→CF₃CF═CH₂, CF₃CF=CH₂+HF→CF₃CF₂CH₃ (245cb). By using a conditioned catalyst, (i.e. a catalyst with lower activity), the unwanted 245cb formation may be reduced.

A conditioned catalyst may be the same catalyst used in the HFO-1234ze(E) manufacturing process as described in Section II but which has already been used for several days, preferably for over 50 days. In practice, a mixture of fresh catalyst and conditioned catalyst can be added to reactor so as to achieve desired 245cb impurity level in the 1234zeE product. These catalysts include any known dehydrofluorination catalyst which may be one or more of fluorinated metal oxides in bulk form or supported, metal halides in bulk form or supported, and carbon supported transition metals, metal oxides and halides. Suitable catalysts non-exclusively include fluorinated chromia (fluorinated Cr₂O₃), fluorinated alumina (fluorinated Al₂O₃), fluorinated mixed metal oxides (e.g., ZnO—Cr₂O₃), metal fluorides (e.g., CrF₃, AlF₃) and carbon supported transition metals (zero oxidation state) such as Fe/C, Co/C, Ni/C, Pd/C.

The conditioned catalyst suitable for reducing the production of HFC-245cb may have a runtime as low as 1 day, 5 days, 10 days, 12 days, 15 days, 20 days, 25 days, 28 days, 30 days, 35 days, 45 days, 50 days, 52 days, 55 days, 60 days, 65 days, 70 days, 75 days, 85 days, 90 days, 95 days, 96 days, 100 days, 110 days, 120 days, 130 days, 140 days, 150 days, 160 days, 170 days, 180 days, 190 days, 200 days, 210 days, 220 days, 225 days, 230 days, 240 days, 250 days, 260 days, 270 days, 280 days, 300 days, 310 days, 320 days, 330 days, 340 days, 350 days, 360 days, or as high as 370 days, 380 days, 390 days, 400 days, 410 days, 420 days, 430 days, 440 days, 450 days, 454 days, 460 days, 470 days, 480 days, 490 days, 500 days, 510 days, 520 days, 530 days, 540 days, 550 days, 560 days, 570 days, 580 days, 590 days, 600 days, or within any range encompassed by any two of the foregoing values as endpoints. For example, the catalyst may have a runtime of 20 to 500 days, 50 to 500 days, or 300 to 500 days.

A catalyst mixture comprising conditioned catalyst blended with fresh catalyst may also be useful for reducing the production of HFC-245cb. The mixture may comprise fresh catalyst in an amount as low as 5 wt. %, 10 wt. %, 15 wt. %, 20 wt. %, 25 wt. %, 30 wt. %, 35 wt. %, 40 wt. %, 45 wt. %, 50 wt. %, or as high as 55 wt. %, 60 wt. %, 65 wt. %, 70 wt. %, 75 wt. %, 80 wt. %, 85 wt. %, 90 wt. %, 95 wt. %, or within any range encompassed by any two of the foregoing values as endpoints, based on the total weight of the catalyst mixture. For example, the catalyst mixture may comprise from 10 wt. wt. % to 90 wt. wt. % of fresh catalyst. The catalyst mixture may also comprise conditioned catalyst in an amount as low as 5 wt. %, 10 wt. %, 15 wt. %, 20 wt. %, 25 wt. %, 30 wt. %, 35 wt. %, 40 wt. %, 45 wt. %, or as high as 50 wt. %, 55 wt. %, 60 wt. %, 65 wt. %, 70 wt. %, 75 wt. %, 80 wt. %, 85 wt. %, 90 wt. %, 95 wt. %, or within any range encompassed by any two of the foregoing values as endpoints, based on the total weight of the catalyst mixture. For example, the catalyst mixture may comprise from 10 wt. % to 90 wt. % of conditioned catalyst, or 50 wt. % of conditioned catalyst.

By employing the described catalyst conditioning methods, the amount of HFC-245cb in the product mixture may be as low as 0 ppm, 0.001 ppm, 0.1 ppm, 0.5 ppm, 1 ppm, 5 ppm, 10 ppm, 15 ppm, 20 ppm, 25 ppm, 30 ppm, 35 ppm, 40 ppm, 45 ppm, 50 ppm, 55 ppm, 60 ppm, 65 ppm, or as high as 70 ppm, 75 ppm, 80 ppm, 85 ppm, 90 ppm, 95 ppm, 100 ppm, 105 ppm, 110 ppm, 115 ppm, 120 ppm, 125 ppm, 130 ppm, 140 ppm, 145 ppm, 150 ppm, or within any range encompassed by any two of the foregoing values as endpoints. For example, the amount of HFC-245cb in the 1234zeE product may be from 0.1 ppm to 100 ppm.

### B. Reactor Temperature

It has also been found that changes to the reactor temperature can influence the production of HFC-245cb. Slight reductions in the reactor's operating temperature may result in significant reductions in HFC-245cb levels with minimal impact HFO-1234ze(E) yield.

The dehydrofluorination reactor may be heated by any means known in the art. For example, an electric heating element, a hot oil, or molten salt may be used.

To reduce the production of HFC-245cb, the reactor temperature may be maintained as low as 10° C., 38° C., 93° C., 149° C., 204° C., 232° C., 260° C., 263° C., 266° C., 271° C., 274° C., 277° C., 279° C., 282° C., 285° C., 288° C., or as high as 291° C., 293° C., 296° C., 299° C., 302° C., 304° C., 307° C., 310° C., 313° C., 316° C., 318° C., 321° C., 324° C., 327° C., 329° C., 332° C., 335° C., 338° C., 341° C., 343° C., or within any range encompassed by any two of the foregoing values as endpoints. For example, the reactor temperature may be from 10° C. to 310° C. or from 288° C. to 310° C.

By employing the described temperature control methods, the amount of HFC-245cb in the product mixture may be as low as 0 ppm, 0.001 ppm, 0.1 ppm, 0.5 ppm, 1 ppm, 5 ppm, 10 ppm, 15 ppm, 20 ppm, 25 ppm, 30 ppm, 35 ppm, 40 ppm, 45 ppm, 50 ppm, 55 ppm, 60 ppm, 65 ppm, or as high as 70 ppm, 75 ppm, 80 ppm, 85 ppm, 90 ppm, 95 ppm, 100 ppm, 105 ppm, 110 ppm, 115 ppm, 120 ppm, 125 ppm, 130 ppm, 140 ppm, 145 ppm, 150 ppm, or within any range encompassed by any two of the foregoing values as endpoints. For example, the amount of HFC-245cb in the product mixture may be from 0.1 ppm to 100 ppm.

### C. Contact Time

It has also been found that changes to the contact time between reactants in the HFO-1234ze(E) process can influence the production of HFC-245cb. Reducing the contact time during the dehydrofluorination step may result in reductions in the production of HFC-245cb.

Contact time may be reduced by a variety of methods including operating at lower pressures for the vapor phase reaction (i.e. lower gas density), operating with less catalyst in the reactor(s), operating at high feed rates of either fresh HFC-245fa or recycle, operating at low single-pass conversion of HFC-245fa to provide a high feed rate of recycle material, adding a diluent to the reactor feed material, increasing temperature (i.e. lower gas density), or adding inert solids to the reactor to effectively reduce the catalyst volume.

To reduce the production of HFC-245cb, the contact time may be reduced to as low as 1 second, 2 seconds, 3 seconds, 4 seconds, 5 seconds, 6 seconds, 7 seconds, 8 seconds, 9 seconds, 10 seconds, 11 seconds, 12 seconds, 13 seconds, 14 seconds, 15 seconds, 16 seconds, 17 seconds, 18 seconds, 19 seconds, 20 seconds, 21 seconds, 22 seconds, 23 seconds, 24 seconds, 25 seconds, 26 seconds, 27 seconds, 28 seconds, 29 seconds, 30 seconds, 31 seconds, 32 seconds, 33 seconds, 34 seconds, 35 seconds, 36 seconds, 37 seconds, 38 seconds, 39 seconds, 40 seconds, 41 seconds, 42 seconds, 43 seconds, 44 seconds, 45 seconds, 46 seconds, 47 seconds, 48 seconds, 49 seconds, 50 seconds, 60 seconds, 70 seconds, 80 seconds, or within any range encompassed by any two of the foregoing values as endpoints. For example, the contact time may be from 1 second to 40 seconds, or from 1 second to 20 seconds.

By employing the described contact time reduction methods, the amount of HFC-245cb in the product mixture may be as low as 0 ppm, 0.001 ppm, 0.1 ppm, 0.5 ppm, 1 ppm, 5 ppm, 10 ppm, 15 ppm, 20 ppm, 25 ppm, 30 ppm, 35 ppm, 40 ppm, 45 ppm, 50 ppm, 55 ppm, 60 ppm, 65 ppm, or as high as 70 ppm, 75 ppm, 80 ppm, 85 ppm, 90 ppm, 95 ppm, 100 ppm, 105 ppm, 110 ppm, 115 ppm, 120 ppm, 125 ppm, 130 ppm, 140 ppm, 145 ppm, 150 ppm, or within any range encompassed by any two of the foregoing values as endpoints. For example, the amount of HFC-245cb in the product mixture may be from 0.1 ppm to 100 ppm.

In some embodiments, the method for reducing 1,1,1,2,2-pentafluoropropane (HFC-245cb) in a trans-1,3,3,3-tetrafluoropropene (HFO-1234ze(E)) manufacturing process comprises dehydrofluorinating 1,1,1,3,3-pentafluoropropane (HFC-245fa) with a catalyst mixture to produce a product mixture comprising trans-1,3,3,3-tetrafluoropropene (HFO-1234ze(E)), cis-1,3,3,3-tetrafluoropropene (HFO-1234ze(Z)), 1,1,1,2,2-pentafluoropropane (HFC-245cb), and hydrogen fluoride (HF), wherein the catalyst mixture comprises from 10 wt. % to 90 wt. % of a catalyst having a runtime of from 50 to 500 days in the HFO-1234ze(E) manufacturing process, based on the total weight of the catalyst mixture.

In some embodiments, the method for reducing 1,1,1,2,2-pentafluoropropane (HFC-245cb) in a trans-1,3,3,3-tetrafluoropropene (HFO-1234ze(E)) manufacturing process comprises dehydrofluorinating 1,1,1,3,3-pentafluoropropane (HFC-245fa) with a catalyst mixture to produce a product mixture comprising trans-1,3,3,3-tetrafluoropropene (HFO-1234ze(E)), cis-1,3,3,3-tetrafluoropropene (HFO-1234ze(Z)), 1,1,1,2,2-pentafluoropropane (HFC-245cb), and hydrogen fluoride (HF), wherein the catalyst mixture comprises about 50 wt. % of a catalyst with a runtime from 300 to 500 days in the HFO-1234ze(E) manufacturing process.

In some embodiments, the method for reducing 1,1,1,2,2-pentafluoropropane (HFC-245cb) in a trans-1,3,3,3-tetrafluoropropene (HFO-1234ze(E)) manufacturing process comprises dehydrofluorinating 1,1,1,3,3-pentafluoropropane (HFC-245fa) with a catalyst mixture to produce a product mixture comprising trans-1,3,3,3-tetrafluoropropene (HFO-1234ze(E)), cis-1,3,3,3-tetrafluoropropene (HFO-1234ze(Z)), 1,1,1,2,2-pentafluoropropane (HFC-245cb), and hydrogen fluoride (HF), wherein the catalyst mixture comprise a dehydrofluorination catalyst selected from the group consisting of fluorinated metal oxides, metal halides, and carbon supported transition metals.

In some embodiments, the method for reducing 1,1,1,2,2-pentafluoropropane (HFC-245cb) in a trans-1,3,3,3-tetrafluoropropene (HFO-1234ze(E)) manufacturing process comprises dehydrofluorinating 1,1,1,3,3-pentafluoropropane (HFC-245fa) with a catalyst mixture to produce a product mixture comprising trans-1,3,3,3-tetrafluoropropene (HFO-1234ze(E)), cis-1,3,3,3-tetrafluoropropene (HFO-1234ze(Z)), 1,1,1,2,2-pentafluoropropane (HFC-245cb), and hydrogen fluoride (HF), followed by a distillation step to recover trans-1,3,3,3-tetrafluoropropene (HFO-1234ze(E)) from the product mixture.

In some embodiments, the method for reducing 1,1,1,2,2-pentafluoropropane (HFC-245cb) in a trans-1,3,3,3-tetrafluoropropene (HFO-1234ze(E)) manufacturing process comprises dehydrofluorinating 1,1,1,3,3-pentafluoropropane (HFC-245fa) with a catalyst mixture to produce a product mixture comprising trans-1,3,3,3-tetrafluoropropene (HFO-1234ze(E)), cis-1,3,3,3-tetrafluoropropene (HFO-1234ze(Z)), 1,1,1,2,2-pentafluoropropane (HFC-245cb), and hydrogen fluoride (HF), wherein the amount of 1,1,1,2,2-pentafluoropropane (HFC-245cb) in the product mixture is from 0.1 ppm to 100 ppm.

In some embodiments, the method for reducing of 1,1,1,2,2-pentafluoropropane (HFC-245cb) in a trans-1,3,3,3-tetrafluoropropene (HFO-1234ze(E)) manufacturing process comprises dehydrofluorinating 1,1,1,3,3-pentafluoropropane (HFC-245fa) with a catalyst mixture to produce a product mixture comprising trans-1,3,3,3-tetrafluoropropene (HFO-1234ze(E)), cis-1,3,3,3-tetrafluoropropene (HFO-1234ze(Z)), 1,1,1,2,2-pentafluoropropane (HFC-245cb), and hydrogen fluoride (HF), wherein the dehydrofluorination step is conducted at a temperature of from 288° C. to 310° C. and the temperature of the dehydrofluorination step is maintained with an electric heating element, a hot oil, or a molten salt.

In some embodiments, the method for reducing 1,1,1,2,2-pentafluoropropane (HFC-245cb) in a trans-1,3,3,3-tetrafluoropropene (HFO-1234ze(E)) manufacturing process comprises dehydrofluorinating 1,1,1,3,3-pentafluoropropane (HFC-245fa) with a catalyst mixture to produce a product mixture comprising trans-1,3,3,3-tetrafluoropropene (HFO-1234ze(E)), cis-1,3,3,3-tetrafluoropropene (HFO-1234ze(Z)), 1,1,1,2,2-pentafluoropropane (HFC-245cb), and hydrogen fluoride (HF), wherein the contact time is from 1 second to 20 seconds and the dehydrofluorination step is conducted at a temperature of from 288° C. to 310° C.

In some embodiments, the method for reducing 1,1,1,2,2-pentafluoropropane (HFC-245cb) in a trans-1,3,3,3-tetrafluoropropene (HFO-1234ze(E)) manufacturing process comprises dehydrofluorinating 1,1,1,3,3-pentafluoropropane (HFC-245fa) with a catalyst mixture to produce a product mixture comprising trans-1,3,3,3-tetrafluoropropene (HFO-1234ze(E)), cis-1,3,3,3-tetrafluoropropene (HFO-1234ze(Z)), 1,1,1,2,2-pentafluoropropane (HFC-245cb), and hydrogen fluoride (HF), wherein the contact time is from 1 second to 20 seconds and the dehydrofluorination step is conducted at a temperature of from 288° C. to 310° C., and further comprising recovering trans-1,3,3,3-tetrafluoropropene (HFO-1234ze(E)) from the product mixture by distillation.

In some embodiments, the method for reducing 1,1,1,2,2-pentafluoropropane (HFC-245cb) in a trans-1,3,3,3-tetrafluoropropene (HFO-1234ze(E)) manufacturing process comprises dehydrofluorinating 1,1,1,3,3-pentafluoropropane (HFC-245fa) with a catalyst mixture to produce a product mixture comprising trans-1,3,3,3-tetrafluoropropene (HFO-1234ze(E)), cis-1,3,3,3-tetrafluoropropene (HFO-1234ze(Z)), 1,1,1,2,2-pentafluoropropane (HFC-245cb), and hydrogen fluoride (HF), wherein the contact time is from 1 second to 20 seconds and the dehydrofluorination step is conducted at a temperature of from 288° C. to 310° C. and the amount of 1,1,1,2,2-pentafluoropropane (HFC-245cb) in the product mixture is from 0.1 ppm to 100 ppm.

### Examples

Throughout the Examples, the data points on HFC-245cb, reactor runtime, temperature, and contact time were determined as follows:

245cb Data: measured via GC in the distillate of the Product Column, which is routed to Product Storage tanks. The Product Column purifies HFO-1234ze(E) and sends HFO-1234ze(Z) and HFC-245fa back to the reactor.

Runtime Data: number of days while fresh HFC-245fa is being fed to the process.

Reactor Temperature: Approximate average reactor temperature as there is a gradient across the reactor due to the endothermic reaction of HFC-245fa to HFO-1234ze(E) and HF.

Contact Time: calculated as Catalyst Volume divided by Volumetric Flowrate of the process flow to the reactor. The catalyst volume is a known number based on the design of the reactor. The volumetric flowrate is based on a calculated density using the ideal gas law and the measured process feed rate to the reactor (i.e. fresh HFC-245fa+recycle).

### Example 1: Catalyst Conditioning

Example 1 demonstrates the effects of catalyst conditioning on HFC-245cb production in the HFO-1234ze(E) process. It was found that the HFC-245cb impurity is produced at levels as high as 1000 ppm when a full batch of fresh fluorinated Cr₂O₃ catalyst was introduced into the reactor. A typical catalyst conditioning curve is set forth in the data in Table 1 below. For the data in Table 1 and 2, the reactor was operating at a temperature between 300-320° C. and a pressure between 5-15 psig. The total catalyst volume of the reactor system was 150-175 ft³ and the total feedrate to the reactor system was 2,000-10,000 lb/hr.

TABLE 1
Effect of Fresh Catalyst Degradation on HFC-245cb Production

| Reactor Runtime (days) | Level of HFC-245cb (ppm) |
|---|---|
| 1 | 1000 |
| 12 | 180 |
| 28 | 120 |
| 52 | 60 |
| 96 | 30 |
| 225 | 15 |
| 454 | 9 |

To ascertain the effect of catalyst conditioning on HFC-245cb production, a mixture of 50% old fluorinated Cr₂O₃ catalyst (with over 300 days of on-stream time) and 50% fresh fluorinated Cr₂O₃ catalyst was used in the HFO-1234ze(E) process reactor. The results are summarized in Table 2 below.

TABLE 2
Effect of 50/50 Used and Fresh Catalyst on HFC-245cb Production

| Reactor Runtime (days) | Level of HFC-245cb (ppm) |
|---|---|
| 1 | 400 |
| 12 | 150 |
| 28 | 100 |
| 52 | 50 |
| 96 | 20 |
| 225 | 10 |
| 454 | 5 |

The results in Table 1 and 2 show that using a conditioned catalyst decreased the level of HFC-245cb production from the outset and helps keep the HFC-245cb levels low as the reactor operates and the catalyst degrades.

### Example 2: Reactor Temperature

Example 2 demonstrates the effects of reducing the reactor temperature on the production of HFC-245cb. The results are summarized in Table 3. For the data in Table 3, the reactor was operating at a pressure between 5-15 psig. The total catalyst volume of the reactor system was 300-350 ft³ and the total feedrate to the reactor system was 4,000-15,000 lb/hr.

TABLE 3
Effect of Temperature on HFC-245cb Production

| Reactor Temperature (° C.) | Level of HFC-245cb (ppm) |
|---|---|
| 313 | 25 |
| 310 | 22 |
| 307 | 18 |
| 304 | 15 |

The results in Table 3 show that decreasing the reactor temperature correlates with an improvement in the production of HFC-245cb.

### Example 3: Contact Time

Example 3 demonstrates the effects of reducing contact time of the process material with the reactor catalyst on HFC-245cb production. The results are summarized in Table 4. For the data in Table 4, the reactor was operating at a temperature between 300-305° C. and a pressure between 5-15 psig. The total catalyst volume of the reactor system was 150-350 ft³ and the total feedrate to the reactor system was 2,000-15,000 lb/hr. The contact time was reduced by removing one of two reactors from service and operating with a high reactor feedrate.

TABLE 4
Effect of Contact Time on HFC-245cb Production

| Catalyst Contact Time (seconds) | Level of HFC-245cb (ppm) |
|---|---|
| 66 | 27 |
| 14 | 4 |

The results in Table 4 show that decreasing the catalyst contact time correlates with an improvement in the production of HFC-245cb.

It should be understood that the foregoing description is only illustrative of the present disclosure. Various alternatives and modifications can be devised by those skilled in the art without departing from the disclosure. Accordingly, the present disclosure is intended to embrace all such alternatives, modifications and variances that fall within the scope of the appended claims.

### ASPECTS

Aspect 1 is a method for reducing 1,1,1,2,2-pentafluoropropane (HFC-245cb) in a trans-1,3,3,3-tetrafluoropropene (HFO-1234ze(E)) manufacturing process comprising: dehydrofluorinating 1,1,1,3,3-pentafluoropropane (HFC-245fa) with a catalyst mixture to produce a product mixture comprising trans-1,3,3,3-tetrafluoropropene (HFO-1234ze(E)), cis-1,3,3,3-tetrafluoropropene (HFO-1234ze(Z)), 1,1,1,2,2-pentafluoropropane (HFC-245cb), and hydrogen fluoride (HF), wherein the catalyst mixture comprises from 10 wt. % to 90 wt. % of a conditioned catalyst having a runtime of from 20 to 500 days in the HFO-1234ze(E) manufacturing process, based on the total weight of the catalyst mixture.

Aspect 2 is the method of Aspect 1, wherein the catalyst mixture comprises 10 wt. % to 90 wt. % of a conditioned catalyst having a runtime of from 50 to 500 days in the HFO-1234ze€ manufacturing process, based on the total weight of the catalyst mixture.

Aspect 3 is the method of Aspect 1 or Aspect 2, wherein the catalyst mixture comprises 50 wt. % of a conditioned catalyst with a runtime from 300 to 500 days in the HFO-1234ze(E) manufacturing process.

Aspect 4 is the method of any one of Aspects 1-3, wherein the catalyst mixture comprise a dehydrofluorination catalyst selected from the group consisting of fluorinated metal oxides, metal halides, and carbon supported transition metals.

Aspect 5 is the method of any one of Aspects 1-4, further comprising recovering trans-1,3,3,3-tetrafluoropropene (HFO-1234ze(E)) from the product mixture by distillation.

Aspect 6 is the method of any one of Aspects 1-5, wherein the amount of 1,1,1,2,2-pentafluoropropane (HFC-245cb) in the product mixture is from 0.1 ppm to 100 ppm.

Aspect 7 is a method for reducing of 1,1,1,2,2-pentafluoropropane (HFC-245cb) in a trans-1,3,3,3-tetrafluoropropene (HFO-1234ze(E)) manufacturing process comprising: dehydrofluorinating 1,1,1,3,3-pentafluoropropane (HFC-245fa) with a catalyst mixture to produce a product mixture comprising trans-1,3,3,3-tetrafluoropropene (HFO-1234ze(E)), cis-1,3,3,3-tetrafluoropropene (HFO-1234ze(Z)), 1,1,1,2,2-pentafluoropropane (HFC-245cb), and hydrogen fluoride (HF), wherein the dehydrofluorination step is conducted at a temperature of from 10° C. to 310° C.

Aspect 8 is the method of Aspect 7, wherein the dehydrofluorination step is conducted at a temperature of from 288° C. to 310° C.

Aspect 9 is the method of Aspect 7 or Aspect 8, wherein the temperature of the dehydrofluorination step is maintained with an electric heating element, a hot oil, or a molten salt.

Aspect 10 is the method of any one of Aspects 7-9, further comprising feeding a reactant mixture comprising 1,1,1,3,3-pentafluoropropane (HFC-245fa) into the dehydrofluorination reaction, wherein the reactant mixture is at a temperature of from 288° C. to 310° C.

Aspect 11 is the method of Aspect 10, wherein the temperature of the reactant mixture is maintained with an electric heating element, a hot oil, or a molten salt.

Aspect 12 is the method of any one of Aspects 7-11, further comprising recovering trans-1,3,3,3-tetrafluoropropene (HFO-1234ze(E)) from the product mixture by distillation.

Aspect 13 is the method of any one of Aspects 7-12, wherein the amount of 1,1,1,2,2-pentafluoropropane (HFC-245cb) in the product mixture is from 0.1 ppm to 100 ppm.

Aspect 14 is a method for reducing 1,1,1,2,2-pentafluoropropane (HFC-245cb) in a trans-1,3,3,3-tetrafluoropropene (HFO-1234ze(E)) manufacturing process comprising: dehydrofluorinating 1,1,1,3,3-pentafluoropropane (HFC-245fa) with a catalyst mixture to produce a product mixture comprising trans-1,3,3,3-tetrafluoropropene (HFO-1234ze(E)), cis-1,3,3,3-tetrafluoropropene (HFO-1234ze(Z)), 1,1,1,2,2-pentafluoropropane (HFC-245cb), and hydrogen fluoride (HF), wherein the contact time in the dehydrofluorination step is from 1 second to 40 seconds.

Aspect 15 is the method of Aspect 14, wherein the contact time is from 1 second to 20 seconds.

Aspect 16 is the method of Aspect 14 or Aspect 15, wherein the dehydrofluorination step is conducted at a temperature of from 288° C. to 310° C.

Aspect 17 is the method of any one of Aspects 14-16, further comprising recovering trans-1,3,3,3-tetrafluoropropene (HFO-1234ze(E)) from the product mixture by distillation.

Aspect 18 is the method of any one of Aspects 15-17, wherein the amount of 1,1,1,2,2-pentafluoropropane (HFC-245cb) in the product mixture is from 0.1 ppm to 100 ppm.

Aspect 19 is a composition comprising trans-1,3,3,3-tetrafluoropropene (HFO-1234ze(E)) as produced by the process according to any one of Aspects 1-18.

Aspect 20 is the composition of Aspect 19, wherein the amount of 1,1,1,2,2-pentafluoropropane (HFC-245cb) in the composition is from 0.1 ppm to 100 ppm.

Aspect 21 is a composition comprising: trans-1,3,3,3-tetrafluoropropene (HFO-1234ze(E)); cis-1,3,3,3-tetrafluoropropene (HFO-1234ze(Z)); 0.1 ppm to 100 ppm of 1,1,1,2,2-pentafluoropropane (HFC-245cb); and and hydrogen fluoride (HF).

What is claimed is:

1. A method for reducing 1,1,1,2,2-pentafluoropropane (HFC-245cb) in a trans-1,3,3,3-tetrafluoropropene (HFO-1234ze(E)) manufacturing process comprising:
dehydrofluorinating 1,1,1,3,3-pentafluoropropane (HFC-245fa) with a catalyst mixture to produce a product mixture comprising trans-1,3,3,3-tetrafluoropropene (HFO-1234ze(E)), cis-1,3,3,3-tetrafluoropropene (HFO-1234ze(Z)), 1,1,1,2,2-pentafluoropropane (HFC-245cb), and hydrogen fluoride (HF),
wherein the catalyst mixture comprises from 10 wt. % to 90 wt. % of a conditioned catalyst having a runtime of from 20 to 500 days in the HFO-1234ze(E) manufacturing process, based on the total weight of the catalyst mixture.

2. The method of claim 1, wherein the catalyst mixture comprises 10 wt. % to 90 wt. % of a conditioned catalyst having a runtime of from 50 to 500 days in the HFO-1234ze(E) manufacturing process, based on the total weight of the catalyst mixture.

3. The method of claim 1, wherein the catalyst mixture comprises 50 wt. % of a conditioned catalyst with a runtime from 300 to 500 days in the HFO-1234ze(E) manufacturing process.

4. The method of claim 1, wherein the catalyst mixture comprise a dehydrofluorination catalyst selected from the group consisting of fluorinated metal oxides, metal halides, and carbon supported transition metals.

5. The method of claim 1, further comprising recovering trans-1,3,3,3-tetrafluoropropene (HFO-1234ze(E)) from the product mixture by distillation.

6. The method of claim 1, wherein the amount of 1,1,1,2,2-pentafluoropropane (HFC-245cb) in the product mixture is from 0.1 ppm to 100 ppm.

7. A method for reducing of 1,1,1,2,2-pentafluoropropane (HFC-245cb) in a trans-1,3,3,3-tetrafluoropropene (HFO-1234ze(E)) manufacturing process comprising:
dehydrofluorinating 1,1,1,3,3-pentafluoropropane (HFC-245fa) with a catalyst mixture to produce a product mixture comprising trans-1,3,3,3-tetrafluoropropene (HFO-1234ze(E)), cis-1,3,3,3-tetrafluoropropene (HFO-1234ze(Z)), 1,1,1,2,2-pentafluoropropane (HFC-245cb), and hydrogen fluoride (HF),
wherein the dehydrofluorination step is conducted at a temperature of from 10° C. to 310° C.

8. The method of claim 7, wherein the dehydrofluorination step is conducted at a temperature of from 288° C. to 310° C.

9. The method of claim 7, wherein the temperature of the dehydrofluorination step is maintained with an electric heating element, a hot oil, or a molten salt.

10. The method of claim 7, further comprising feeding a reactant mixture comprising 1,1,1,3,3-pentafluoropropane (HFC-245fa) into the dehydrofluorination reaction, wherein the reactant mixture is at a temperature of from 288° C. to 310° C.

11. The method of claim 10, wherein the temperature of the reactant mixture is maintained with an electric heating element, a hot oil, or a molten salt.

12. The method of claim 7, further comprising recovering trans-1,3,3,3-tetrafluoropropene (HFO-1234ze(E)) from the product mixture by distillation.

13. The method of claim 7, wherein the amount of 1,1,1,2,2-pentafluoropropane (HFC-245cb) in the product mixture is from 0.1 ppm to 100 ppm.

14. A method for reducing 1,1,1,2,2-pentafluoropropane (HFC-245cb) in a trans-1,3,3,3-tetrafluoropropene (HFO-1234ze(E)) manufacturing process comprising:
dehydrofluorinating 1,1,1,3,3-pentafluoropropane (HFC-245fa) with a catalyst mixture to produce a product mixture comprising trans-1,3,3,3-tetrafluoropropene (HFO-1234ze(E)), cis-1,3,3,3-tetrafluoropropene (HFO-1234ze(Z)), 1,1,1,2,2-pentafluoropropane (HFC-245cb), and hydrogen fluoride (HF),
wherein the contact time in the dehydrofluorination step is from 1 second to 40 seconds.

15. The method of claim 14, wherein the contact time is from 1 second to 20 seconds.

16. The method of claim 14, wherein the dehydrofluorination step is conducted at a temperature of from 288° C. to 310° C.

17. The method of claim 14, wherein the amount of 1,1,1,2,2-pentafluoropropane (HFC-245cb) in the product mixture is from 0.1 ppm to 100 ppm.

18. A composition comprising trans-1,3,3,3-tetrafluoropropene (HFO-1234ze(E)) as produced by the process according to claim 1.

19. The composition of claim 18, wherein the amount of 1,1,1,2,2-pentafluoropropane (HFC-245cb) in the composition is from 0.1 ppm to 100 ppm.

20. A composition comprising:
trans-1,3,3,3-tetrafluoropropene (HFO-1234ze(E));
cis-1,3,3,3-tetrafluoropropene (HFO-1234ze(Z));
0.1 ppm to 100 ppm of 1,1,1,2,2-pentafluoropropane (HFC-245cb); and
and hydrogen fluoride (HF).
